(19) United States
(12) Patent Application Publication
(10) Pub. No.: US 2014/0271847 A1
(43) Pub. Date: Sep. 18, 2014

(54) FORMULATIONS AND METHODS FOR RAPID PENILE ERECTIONS

(71) Applicant: SatisPharma, LLC, Boulder, CO (US)

(72) Inventors: Ray L. Hauser, Boulder, CO (US); BoLin Zheng, Boulder, CO (US)

(21) Appl. No.: 14/210,280

(22) Filed: Mar. 13, 2014

Related U.S. Application Data
(60) Provisional application No. 61/779,517, filed on Mar. 13, 2013.

(51) Int. Cl. A61K 9/00; A61K 31/4985; A61K 36/87; A61K 33/40; A61K 31/198; A61K 31/519; A61K 31/53

(52) U.S. Cl. CPC A61K 9/0056; A61K 31/519; A61K 31/4985; A61K 31/53; A61K 33/40; A61K 31/198; A61K 36/87
USPC 424/464; 544/262; 514/252.16; 544/343; 514/250; 544/184; 514/243; 424/613; 424/766

(57) ABSTRACT

There is disclosed a formulation for creating a rapid penile erection within 30 minutes. In an embodiment, a lozenge has a preparation configured to create a rapid penile erection. The preparation is effective to create the rapid penile erection with sildenafil citrate in a range of 50-100 mg. The preparation is effective to create the rapid penile erection with tadalafil in a range of 5-40 mg. The preparation is effective to create the rapid penile erection with vardenafil hydrochloride in a range of 5-40 mg. In various embodiments, the formulation includes a solvent configured to permeate skin. Other embodiments are also disclosed.

### REFERENCE TO PENDING PRIOR PATENT APPLICATION

This application claims the benefit under 35 U.S.C. 119 (e) of U.S. Provisional Patent Application No. 61/779,517, filed Mar. 13, 2013 by Ray L. Hauser, et al., for “FORMULATIONS AND METHODS FOR RAPID PENILE ERECTIONS,” which patent application is hereby incorporated herein by reference.

### BACKGROUND

Erectile dysfunction (ED) is the inability for a man to obtain a firm and lasting erection of the penis. This can be due to any of a variety of causes, including old age, physical injuries and psychological factors. Lack of satisfying sexual experiences can destroy marriages. Developments of ED medications over the past 20 years have been very helpful in providing satisfaction to both partners.

The human body has an active/reactive mechanism for controlling erection of the penis. The active mechanisms involve physical stimulation, emotional stimulation, or both, as related to the penis such that blood vessels relax and permit sufficient flow of blood to expand the vessels, increasing volume and rigidity. Technically, the physiologic mechanism of erection of the penis involves release of nitric oxide in the corpus cavernosum during sexual stimulation. The nitric oxide activates the enzyme guanylate cyclase, which results in increased levels of cyclic guanosine monophosphate, producing smooth muscle relaxation in the corpus cavernosum and allowing inflow of blood.

Concurrently, phosphodiesterase type 5 in the penis acts to decrease the amount of cyclic guanosine monophosphate, which decreases the amount of blood in the penis, prompting a normal flaccid condition.

Sildenafil citrate and other treatments for erectile dysfunction enhance the effect of nitric oxide by inhibiting the effects of the cyclic guanosine monophosphate for a sufficient duration to permit good erection and stiffness for satisfactory coitus for the participants.

Sildenafil citrate is sold globally under the trade name Viagra® and is described in U.S. Pat. Nos. 5,482,941 and 6,469,012, assigned to Pfizer. Cialis® is another treatment for erectile dysfunction, technically known as tadalafil, and U.S. Pat. Nos. 5,859,006 and 6,140,329 are assigned to Eli Lilly. Levitra® is a third treatment for erectile dysfunction, technically known as vardenafil hydrochloride, and U.S. Pat. Nos. 6,362,178 and 7,696,206 are assigned to Bayer. The three treatments have somewhat comparable objectives, but doses are different, and effectiveness may vary from individual to individual (e.g., 25 to 200 mg of Viagra® brand sildenafil). The Cialis® brand tadalafil dosage was originally 20 mg per occasion, but is now also sold as 5 mg daily tablets for presumed “ready at any time.” The Levitra® dosage is usually 10 or 20 mg per occasion, and Bayer has more recently added a lozenge formulation with 10 mg of vardenafil hydrochloride (trade name Staxyn®). Bayer recommends taking this product one hour prior to intercourse. The term, “erectile dysfunction medication” is used in this patent application to refer to a formulation containing sildenafil citrate, tadalafil, or vardenafil or other pharmaceuticals that have similar functions inhibiting the effects of phosphodiesterase type 5.

Normally, an erectile dysfunction pill is swallowed and the active chemicals are absorbed through the intestines into the blood stream. Pfizer reports that the 100 mg pill gives an average maximum concentration of sildenafil citrate of 450 nanograms/ml in blood plasma at about 1 hour after taking the pill, and this is the optimum time for proceeding with sexual intercourse. Pfizer reports that an average of about 41% of the sildenafil citrate reaches the blood after the pill is swallowed, with 59% of the active ingredient passing through the body, mainly in the feces.

As the Pfizer patents have not had international validity, there are a number of knock-offs for their Viagra® product, with variations in formulations. Pharmaceutical suppliers have marketed lozenge tablets for sublingual use and solutions for absorption through the mucosal surfaces of the mouth. U.S. Pat. No. 6,200,592 describes a nasal spray containing sildenafil citrate with unsupported claims of absorption and erectile effectiveness within 5 minutes.

When active medications are absorbed in the mucosal surfaces of the mouth or nose, the delivery into the blood stream is usually in the 90-95% range if saliva generation and swallowing are minimized. Thus a lozenge with 50 mg of active ingredient may be as effective as a swallowed pill with 100 mg. Some of the suppliers of products for erectile dysfunction claim “instant” response. Absorption of the chemicals in the mouth may occur within about 5 minutes, but the effective time to erection is usually 15 to 30 minutes after taking the lozenge. This implies that it takes some time for sufficient ED stimulant to be absorbed within the penis to overcome the pre-existing reservoir of guanosine monophosphate and to generate nitrogen oxide.

Responses of individuals to ED medications may vary widely from one to another and from time to time. The nature and intensity of emotional and physical stimulation are significant factors, and the health of the individual can be significant. Fatigue, time of day, time since last coitus or ejaculation, and blood circulation are factors influencing response. One particular subject had no response to Cialis®. His heart damage and occluded artery limit blood circulation to about 50%. Thus this particular subject is not a good benchmark for measuring the effectiveness of each formulation; this particular subject experiences a normal dose of 100 mg Viagra® or 10 mg Levitra® usually takes about 2 hours to become effective and then remains effective for about 8 hours (double the usual times for a person with normal blood circulation). Nevertheless, this subject has achieved erection and cotius within 30 minutes using some formulations of this invention.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In an embodiment, there is provided a formulation for creating a rapid penile erection, the formulation comprising a preparation configured to create a rapid penile erection within 30 minutes.

In one embodiment, the preparation is effective to create the rapid penile erection with sildenafil citrate in a range of 50-100 mg.

In another embodiment, the preparation is effective to create the rapid penile erection with tadalafil in a range of 5-40 mg.

In still another embodiment, the preparation is effective to create the rapid penile erection with vardenafil hydrochloride in a range of 5-40 mg.

In an embodiment, the preparation includes a solvent configured to permeate skin.

Other embodiments are also disclosed.

Additional objects, advantages and novel features of the technology will be set forth in part in the description which follows, and in part will become more apparent to those skilled in the art upon examination of the following, or may be learned from practice of the technology.

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

The erectile dysfunction medication must transfer from its formulation of a lozenge or solution first into the blood stream and then into the penis.

Various embodiments use formulations that can be absorbed rapidly through mucosal membranes. Exemplary embodiments address the flow of saliva outward from the mucosal surfaces and the flow of the active ingredients into the mucosal surfaces where these active ingredients can be absorbed into the blood stream.

In order for an ED medication to be absorbed into mucosal tissues, it must first be dissolved in a medium of transport. Sildenafil citrate is soluble in water at pH=7.0 to the extent of 3.5 mg/ml, but at pH=4, it is soluble about triple this concentration. Sildenafil citrate is also more soluble in ethanol than water and in propylene glycol than in water. Sildenafil citrate has surprisingly high solubility in nicotine. Tadalafil is freely soluble in water. Vardenafil is freely soluble in water and in alcohols.

Sildenafil citrate has a bitter taste and this taste is a deterrent to its use as a lozenge or solution in the mouth with its myriad of taste buds. A taste maskant is needed to overcome this objection. Citric acid is an effective taste maskant that can concurrently be used at a concentration to provide a pH equal to 4 to allow greater dissolution of the active ingredient in an acidic solution than pH neutral water. The sourness of citric acid can be mollified by a sweetener.

A variety of excipients are known to assist mass transfer of active pharmaceuticals through the skin, but there are few publications related to transfer through mucous membranes. The dry human skin has an oily content (sebum) and is an intentional barrier to transfer of water in either direction, except for sweat glands which are mostly unidirectional in the flow of water. Surfactants are sometimes used to assist transfer of material through external skin and surfactants may be of some benefit for transfer through mucous membranes of the mouth. A paper by D. W. Osborne & J. J. Henke lists about 400 permeants in their paper, “Skin Penetration Enhancers Cited in Technical Literature,” Pharmaceutical Technology, November 1997, pp 85-91, but only a few can both dissolve sildenafil citrate and assist its permeation through mucous membranes. Glycerol, propylene glycol, isopropyl myristate, and nicotine have these attributes.

Some permeants are known to have a residual effect, as if they effectively open doors in membranes that permit a stream of other ingredients to follow through. Isopropyl myristate is one such permeant, and nicotine may perform the same function. This effect can be enhanced by incorporating such a permeant in a lozenge or solution of the active ingredient.

Surprisingly, nicotine has been found to be a very effective solvent for sildenafil citrate (as noted below), and its very rapid absorption through oral mucosa is experienced by cigarette smokers with the first puff. One milligram of nicotine per dose is often used with formulations such as Nicorette-NS for people attempting to break the smoking habit, and nicotine can be an effective component of an ED lozenge or solution.

A Viagra® pill delivering 100 mg of sildenafil citrate weighs about 620 mg. Much of this weight is excipients, particularly microcrystalline cellulose, which assists both compaction and then physical disintegration of the pill in the intestines. This pill has a surface area about 3.2 sq. cm. For rapid dissolution of a lozenge in the mouth, a minimal amount of inert material is desired, formed with a maximum surface area. In an exemplary embodiment, lozenges with 50-100 mg sildenafil citrate have been made with total weight in the range of 300 to 400 milligrams. These lozenges have been formed in pills about 1.9 cm diameter and 0.12 cm thick with surface area of 5.4 cm². These lozenges have also been formed in pills having open-cell porous structure to absorb saliva quickly, providing rapid dissolution.

A polymeric binder may be used for making the lozenges. Exemplary polymeric binders may include, but are not limited to, polyvinylpyrrolidone, polyvinyl alcohol, polyethylene glycol, pullulan, methyl cellulose, hydroxypropyl methyl cellulose, polyethylene oxide, or other pharmaceutical grade water-soluble polymers. A small amount of plasticizer is of value, which may be a permeant that is capable of dissolving the erectile dysfunction medication. Hot-melt extrusion may be used to cast a thin sheet of the formulation onto an adhesive substrate surface. Discs or wafers of the medication can then be die-cut. Hexagonal wafers are a prospective geometry for lozenges. Lozenges cast from solution are also porous.

In one embodiment, the directionality of mass transfer at mucosal surfaces has been addressed. To dissolve a lozenge, sufficient saliva is needed, particularly from the sub-lingual ducts; but to transfer this active solution into the blood stream, this new saliva solution must then permeate into the mucosal surfaces of the cheeks and tongue. Osmotic pressure differences are the driving forces for each mass transfer. Lozenges have been made with two and with three layers. The core of such a lozenge may contain a small amount (about 0.5 mg/pill) of atropine which decreases flow of saliva and prompts a “dry mouth” feeling. One or both outer surfaces of the lozenge may contain acetyl choline hydrochloride (about 0.1 mg/pill), which stimulates the production of saliva. When only one such outer surface is a different color from the bulk of the lozenge, supplier's instructions can state that the lozenge should be placed under the tongue with this surface on the downward side, toward the saliva ducts. Thus stimulation of saliva is accomplished quickly for dissolution. Then the lozenge can be moved about to contact inner cheeks, tongue and roof of mouth to maximize absorption into these “dry” membranes. A distinct flavoring within the acetyl choline hydrochloride layer can be an indicator for time to move the lozenge. One or two coatings can be applied to the cast lozenge and/or to the substrate surface for an embodiment. One side can have the saliva enhancement; the other side can have the saliva restriction and a permeation enhancement. The two sides can have different colors and flavors.

In one embodiment, the active medication is added as a very fine powder or is pre-dissolved in a solvent in order to obtain molecular-scale dispersion throughout the lozenge. When the medication is added as a solution to a lozenge formulation the solvent is evaporated and the mix can then be compacted or melt-extruded.

In another embodiment, the lozenge can be formulated with ingredients to prompt rapid disintegration, thus rapid dissolution in saliva within the mouth. Acid-base reactions such as citric acid-sodium bicarbonate cause an effervescence prompting such disintegration. Super-disintegrants can also be used, such as hydroxypropyl cellulose.

In another embodiment, the lozenge can be formulated and produced with a foamed or porous structure such that it starts with a large total surface area for contact and rapid dissolution in saliva. This structure can be accomplished by lyophilization, by simple evaporation of solvent, or by addition of chemical blowing agents, gas-producing reactions, or pressurized gases during extrusion.

Solutions and dispersions of ED medications have also been formulated and found to be effective. These are preferably less than 10 ml total volume, with the active ingredient dissolved in an effective mixture of water, propylene glycol, glycerol, citric acid, and other ingredients, including atropine in one embodiment.

Properly formulated lozenges can dissolve in the mouth within three minutes, and properly formulated solutions can be absorbed by oral tissues within four minutes, as indicated by loss of flavoring within the product.

The transfer of active pharmaceutical from blood to the penis can be enhanced by another embodiment, short-term use of a vacuum chamber to enlarge the penis after having taken the ED medication. Phosphodiesterase type 5 (which prompts relaxation of the penis) is almost unique to this organ of the body, and only a small amount is normally present in the penis, sufficient to maintain a normally flaccid condition and to relax the penis after a brief stimulation toward erection. When blood brings in an active erectile dysfunction medication, such as sildenafil citrate, and the size of the penis increases due to emotional or physical stimulation, the amount of the erectile dysfunction medication increases in proportion to the amount of blood that is present. This process normally takes an estimated 20-30 minutes after an appropriate concentration of the medication is reached in the blood, depending upon the amount of external stimulation. However, this process can be enhanced within 1-2 minutes by use of a vacuum device to expand the penis. A vacuum in the range of 100-200 mm Hg can expand the penis within a cylinder to double its length and diameter very quickly. This 5 to 8-fold increase of blood can quickly provide a sufficient quantity of the erectile dysfunction medication to overwhelm the phosphodiesterase type 5 that is present, and a lasting erection can be accomplished within a few minutes after taking the medication in the mouth. An “erection ring” is generally recommended when vacuum is used without medication; a rubber ring is sometimes slipped from the cylinder onto the base of the penis before release of the vacuum to prevent blood from flowing back into the venous system. Such a ring is seldom effective and it may not be needed when the medication has taken effect prior to using the vacuum pump and if arousal conditions are in effect. This combined effect can be faster and can be much more satisfying to the partners than waiting an hour for the Viagra® pill alternative ED medications.

Another embodiment may include the addition of a small amount of nitrate, such as thiamine, in the formulation. Nitrates in conjunction with ED treatments are generally forbidden by the FDA, as they may cause an excessive drop in blood pressure. However, an amount of nitrate in a daily dosage of multi-vitamins or the amount of nitrite in a slice of salami should be acceptable and this can provide an additional stimulation to nitric oxide production and rapidity of erection.

Another embodiment includes the addition of a nitric oxide precursor such as l-arginine in the formulation, along with an ED medication. Addition of a peroxide along with arginine may further accelerate penile expansion and firmness.

Another embodiment includes the addition of yohimbine (a natural bark extract) in the formula.

Another embodiment includes the addition of extracts from grape seeds, known to be a vasodilator and reducer of systolic blood pressure.

Another embodiment includes the addition of papaverine, a known vasodilator, in the formulation.

Another embodiment may include the incorporation of an aphrodisiac in the formulation, along with an ED medication, e.g., sildenafil citrate, tadalafil or vardenafil.

An exemplary aphrodisiac is an extract from a root vegetable known as maca, technically known as lepidium miyenii. The extract is described by U.S. Pat. Nos. 6,267,995 and 6,428,824 by Zheng et al, assigned to Pure World Botanicals, Inc, South Hackensack N.J. It is described in “Imidazole Alkaloids from Lepidium Meyenii” in J. Nat. Prod. 2003, 66 pp 1101-3.

The medical combination may be in a pill form for swallowing, a lozenge for dissolution in the mouth, or a solution for absorption in the mouth. Compositions and method of treatment for erectile dysfunction are disclosed. The medical combination of ED stimulant with aphrodisiac may include additives to enhance, to restrict, or to first enhance and then restrict, production of saliva within the mouth. Maca aphrodisiac has been found to be an effective binder when compressed and it can reduce the need for conventional binding excipients in a pill or lozenge.

Various embodiments may include addition of sweeteners, flavorings, stabilizers and/or colorants to the formulated product.

### Example 1

A series of experiments were performed in order to learn the solubilities of ED medications in solvents known to permeate the skin safely. Results were as follows:

| Wt. of sildenafil | | |
|---|---|---|
| citrate, mg | Solvent | Result |
| 35 mg | glycerin, 10 ml + | dispersion |
| | citric acid, 150 mg | |
| 35 mg | triacetin 10 ml | dispersion |
| 35 mg | Propylene glycol, 10 ml | fully soluble |
| 70 mg | propylene glycol, 10 ml | mostly soluble |
| 35 mg | ethanol, 190 proof, 10 ml | fully soluble |
| 70 mg | ethanol, 190 proof, 10 ml | fully soluble |
| 35 mg | isopropyl myristate, 10 ml | mostly soluble |
| 35 mg | diethylene glycol | slightly soluble |
| | monoethyl ether, 10 ml | |
| 49 mg | nicotine, 2 ml | fully soluble |

### Example 2

### QK 2

A formulation was prepared with a Pfizer Viagra® 100 mg pill; plus water, 6.7 ml; sucrose 400 mg; citric acid, 50 mg; and polyvinylpyrrolidone (MW 1,300,000 Daltons) 200 mg. This slurry was cast into four cavities in a silicone rubber mold (penny-size) and was allowed to dry fully. The resulting lozenges had a slight bittersweet taste and dissolved sub-lingually within 4 minutes.

### Example 3

### QK 29

An ethanol (190 proof) extraction of a 100 mg Viagra pill having 72 mg of solids in 1 ml was added to water, 0.75 ml and citric acid, 4 mg. This mixture was taken as a solution and held in the mouth for about 1 minute. It had a bitter taste. With moderate physical simulation, a hard penis was reached within 8 minutes and could be regained at about 6 hours later.

### Example 4

### QK 39AB

A formulation was prepared with an alcoholic extract of one 100 mg. Viagra pill evaporated to dryness; propylene glycol 2.4 ml; glycerin 2.4 ml; sucrose 471 mg; citric acid 30 mg; polyvinylpyrrolidone (MW 30,000) 100 mg, nicotinic acid 100 mg; citric acid 36 mg. A teaspoon of this solution was taken and it was absorbed through mucous membranes within 3 minutes. It was not bitter, but an excess of saliva was formed that was difficult to avoid swallowing. The penis was hard within 15 minutes with minimal stimulation.

### Example 6

### QK 80

Five liquid doses were made using 175 mg of sildenafil citrate; 190 proof alcohol, 3 ml; water 10 ml; lemon flavor 1.25 ml; 0.2% nicotine in water 2.5 ml (5 μg); acetylcholine 8 mg; propylene glycol 10 ml; and ascorbic acid 50 mg. This solution had a pH about 3.5. Its taste was acceptable and little saliva formed in the mouth. A teaspoon of the solution was fully absorbed in the mouth within 8 minutes and good firmness was obtained in about 20 minutes.

### Example 7

### QK 99

Five lozenges were made using sildenafil citrate 350 mg; Maca Pure® 2.25 gm; ethanol 6 ml; water 2 ml; sodium benzoate preservative 38 mg; and aspartame sweetener 38 mg. Components were dissolved and dispersed by heating to 150° F. Solution was poured into nickel-size cavities in a silicone mold and allowed to evaporate. The lozenge had a bitter taste.

### Example 8

### QK 102

Two vardenafil 20 mg pills were ground in a mortar-pestle, to which was added acetylcholine 40 mg; powdered sugar 200 mg; and Maca Pure® 900 mg. This mix was pressed into four molds 0.5″ diameter, forming a cohesive pill about 0.16″ thick (4.1 mm). The taste of this lozenge was acceptable but not sweet. The lozenge dissolved sub-lingually within 6 minutes and the penis reached an effective size and firmness after about 10 minutes of foreplay followed by good orgasms.

### Example 9

### QK 117

A formulation was prepared using 10 mg of vardenafil extracted from a commercial source to which was added Maca Pure® 450 mg, Kroger non-sugar sweetener 50 mg; and ethanol 0.5 ml. This was cast in a silicone rubber mold and dried. The lozenge dissolved sublingually within 90 seconds and the penis was firm within 12 minutes.

### Example 10

### QK 130

Eight Cialis® pills listed as 5 mg of active ingredient each were ground by mortar and pestle and added to 10 ml of ethanol and 10 ml of methylene chloride solvents. The mix was sonicated for 30 minutes, decanted and filtered through a glass fiber filter media. Water was added, 50 ml, to the filtrate causing a phase separation with about 10 ml of white dispersion in the bottom layer. This was dried to a weight of 12 mg and was added to 85 mg of Maca Pure®; 60 mg of sucralose, and 7.5 ml of ethanol. The slurry was cast into two nickel-size molds, one lozenge was pressed in a ¾″ diameter mold.

### Example 11

### QK 132

A porous lozenge was made by casting and its density was compared to that of a pressed lozenge of the same formulation. This consisted of vardenafil hydrochloride 92 mg; gelatinized maca 1700 mg; ethanol 2 ml and sucralose 120 mg. The casting in a quarter-size mold had a density of 0.48 gm/cc, and the same formulation pressed in a ¾″ diameter mold had a density of 1.02 gm/cc. This indicated that the dried casting contained about 53% void volume, which means that it had a large surface area conducive toward rapid dissolution in saliva.

### Example 12

### QK 141

Two lozenges were prepared using sildenafil citrate 200 mg; l-arginine 800 mg; sucralose 100 mg; and water 800 mg. The slurry was cast into two silicone rubber molds and allowed to dry. A lozenge fully dissolved sub-lingually within 8 minutes and had a suitable sweet taste.

### Example 13

### QK 142

Two lozenges were prepared as in Example 12, but with the addition of 100 mg of polyvinylopyrollidone (MW=30,000) to provide extra binding strength.

### Example 14

### QK 145

A lozenge was prepared using 20 mg of vardenafil hydrochloride extracted from commercial pills with 50 ml of methanol, to which was added l-arginine 400 mg, sucralose 25 mg, Twinlabs Yohimbe (bark extract) 400 mg. The dried and pressed product gave a lozenge that dissolved sub-lingually within 5 minutes and gave sufficient firmness for penetration within 25 minutes.

### Example 15

### QK 146

A lozenge was prepared using 20 mg of vardenafil hydrochloride extracted from commercial pills with 50 ml of methanol plus l-arginine 400 mg; sucralose 25 mg; and 250 mg of grape seed extract. The cast pill was 2.4 mm diameter, 2.3 mm thick and it weighed 642 mg.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

1. A formulation for creating a rapid penile erection, the formulation comprising a preparation configured to create a rapid penile erection within 30 minutes.

2. The formulation of claim 1, wherein the preparation is effective to create the rapid penile erection with sildenafil citrate in a range of 50-100 mg.

3. The formulation of claim 1, wherein the preparation is effective to create the rapid penile erection with tadalafil in a range of 5-40 mg.

4. The formulation of claim 1, wherein the preparation is effective to create the rapid penile erection with vardenafil hydrochloride in a range of 5-40 mg.

5. The formulation of claim 1, wherein the preparation is effective to create the rapid penile erection with a combination of ED medications.

6. The formulation of claim 1, wherein the preparation has a total weight in a range of 300 to 400 milligrams.

7. The formulation of claim 1, wherein the preparation is configured for oral administration to a patient and the preparation is configured for dissolution within the mouth.

8. The formulation of claim 1, wherein the preparation includes a taste maskant.

9. The formulation of claim 8, wherein the maskant is citric acid.

10. The formulation of claim 9, wherein the citric acid has a pH of 4 to allow greater dissolution of sildenafil citrate.

11. The formulation of claim 1, wherein the preparation is formed in a lozenge having a size of about 1.9 cm diameter and 0.12 cm thick with surface area of 5.4 cm².

12. The formulation of claim 11, wherein the lozenge has an open-cell porous structure to absorb saliva quickly, providing rapid dissolution.

13. The formulation of claim 1, wherein the preparation is formed in a solution and the preparation is configured for oral administration to a patient as the solution.

14. The formulation of claim 1, wherein the formulation further includes an aphrodisiac.

15. The formulation of claim 14, wherein the aphrodisiac is maca aphrodisiac and provides an effective binder when compressed so as to obviate a need for conventional excipients in a pill or lozenge.

16. The formulation of claim 1, further comprising a nitrate.

17. The formulation of claim 16, wherein the nitrate is thiamine.

18. The formulation of claim 1, further comprising a nitric oxide precursor.

19. The formulation of claim 18, wherein the nitric oxide precursor is l-arginine.

20. The formulation of claim 1, further comprising a peroxide, and wherein the peroxide together with l-arginine is configured to accelerate penile expansion and firmness.

21. The formulation of claim 1, further comprising yohimbine.

22. The formulation of claim 1, further comprising an extract from grape seeds, wherein the extract is a vasodilator and configured to reduce systolic blood pressure.

23. The formulation of claim 1, further comprising papaverine.

24. The formulation of claim 1, further comprising a solvent configured to permeate skin.

25. The formulation of claim 14, wherein the formulation further includes additives configured to at least one of (1) enhance production of saliva within the mouth, (2) restrict production of saliva within the mouth, and (3) first enhance production of saliva within the mouth and then restrict production of saliva within the mouth.

26. The formulation of claim 1, wherein the preparation is effective to create the rapid penile erection with sildenafil citrate in a range of 50-200 mg.
